(19) United States
(12) Patent Application Publication
(10) Pub. No.: US 2025/0072839 A1
(43) Pub. Date: Mar. 6, 2025

(54) SYSTEM AND METHOD OF REAL TIME MULTI-SENSOR SIGNAL CORRECTION

(71) Applicant: HAPPY HEALTH, INC., Austin, TX (US)

(72) Inventors: Conrad W. Rosenbrock, Provo, UT (US); David E. Clift-Reaves, Austin, TX (US); Arnulf Graf, Pasadena, CA (US)

(21) Appl. No.: 18/822,147

(22) Filed: Aug. 31, 2024

Related U.S. Application Data
(60) Provisional application No. 63/579,986, filed on Sep. 1, 2023.

(51) Int. Cl. A61B 5/00; G01D 18/00

(52) U.S. Cl. CPC A61B 5/7225; G01D 18/00

(57) ABSTRACT

A computer-implemented method for correcting a plurality of sensor streams in real time is disclosed. The computer-implemented method includes (i) detecting a deviant in a sensor stream of the plurality of sensor streams based upon a distribution of sensor signal values in the sensor stream; (ii) detecting a gap or a period of missing sensor data based upon deviation in a sensor signal sample rate; (iii) performing adaptive interpolation for sensor signal values across the detected gap or the period of missing sensor data; (iv) performing adaptive signal correction by suppressing derivative values of the detected deviant; (v) performing adaptive drift correction to handle asymmetries in a sensor signal during a rise and fall of the sensor signal; and (vi) generating the sensor stream with corrected sensor signal values.

### CROSS-REFERENCE TO RELATED APPLICATION

This application is a nonprovisional and claims the benefit under 35 U.S.C. § 119 (e) of U.S. Provisional Patent Application No. 63/579,986, filed Sep. 1, 2023, the contents of which are incorporated herein by reference as if fully disclosed herein.

### TECHNICAL FIELD

Embodiments described herein generally relate to a system and a method for real-time corrections of signals from multiple sensors, and in particular, real-time corrections of signals from multiple wearable sensors for missing sensor signals and/or removal of artifacts introduced by environmental factors.

### BACKGROUND

Wearable electronic devices such as smart watches, fitness trackers, glucose trackers, etc., include sensors for continuous monitoring of physiological and/or biological signals of a user when a wearable electronic device is worn by the user. The sensors included in the wearable electronic device monitor health-related signals such as step count, heart rate, blood glucose level, blood pressure, respiratory rate, galvanic skin conductance and many others. Sensor data, based upon the monitored signals, helps the user to manage health status and empower the user in self-management of chronic disease conditions for diabetes, obesity, and cardiovascular disease by incorporating certain lifestyle changes. Accordingly, self-monitoring with the wearable electronic device including sensors for continuous monitoring of physiological and/or biological signals improves the overall quality of life and reduce health care costs. However, real-time analysis of the sensor data from the sensors in the wearable electronic device is challenging due to artifacts introduced in the sensor data or missing sensor data due to environmental factors including motion, humidity, temperature, and other environmental factors.

This section is intended to introduce the reader to various aspects of art that may be related to various aspects of the present disclosure described or claimed below. This description is believed to be helpful in providing the reader with background information to facilitate a better understanding of the various aspects of the present disclosure. Accordingly, it should be understood that these statements are to be read in this light and not as admissions of prior art.

### SUMMARY

In one aspect, a computer-implemented method for correcting a plurality of sensor streams in real time is disclosed. The computer-implemented method includes (i) detecting a deviant in a sensor stream of the plurality of sensor streams based upon a distribution of sensor signal values in the sensor stream; (ii) detecting a gap or a period of missing sensor data based upon deviation in a sensor signal sample rate; (iii) performing adaptive interpolation for sensor signal values across the detected gap or the period of missing sensor data; (iv) performing adaptive signal correction by suppressing derivative values of the detected deviant; (v) performing adaptive drift correction to handle asymmetries in a sensor signal during a rise and fall of the sensor signal; and (vi) generating the sensor stream with corrected sensor signal values.

In another aspect, a system for correcting a plurality of sensor streams in real time is disclosed. The system includes at least one non-transitory memory storing instructions, and at least one processor communicatively coupled with the at least one non-transitory memory and configured to execute the instructions to perform operations including (i) detecting a deviant in a sensor stream of the plurality of sensor streams based upon a distribution of sensor signal values in the sensor stream; (ii) detecting a gap or a period of missing sensor data based upon deviation in a sensor signal sample rate; (iii) performing adaptive interpolation for sensor signal values across the detected gap or the period of missing sensor data; (iv) performing adaptive signal correction by suppressing derivative values of the detected deviant; (v) performing adaptive drift correction to handle asymmetries in a sensor signal during a rise and fall of the sensor signal; and (vi) generating the sensor stream with corrected sensor signal values.

In yet another aspect, a non-transitory computer-readable media storing instructions thereon is disclosed. The instructions, when, executed by at least one processor of at least one computing device, cause the at least one computing device to correct a plurality of sensor streams in real time by performing operations including (i) detecting a deviant in a sensor stream of the plurality of sensor streams based upon a distribution of sensor signal values in the sensor stream; (ii) detecting a gap or a period of missing sensor data based upon deviation in a sensor signal sample rate; (iii) performing adaptive interpolation for sensor signal values across the detected gap or the period of missing sensor data; (iv) performing adaptive signal correction by suppressing derivative values of the detected deviant; (v) performing adaptive drift correction to handle asymmetries in a sensor signal during a rise and fall of the sensor signal; and (vi) generating the sensor stream with corrected sensor signal values.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated examples may be incorporated into any of the above-described aspects, alone or in any combination.

### BRIEF DESCRIPTION OF DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1A illustrates an example wearable electronic device;

FIG. 1B illustrates an example block diagram of the wearable electronic device shown in FIG. 1A;

FIG. 2 illustrates an example live memory weighting (LMW) in real time;

FIG. 3 illustrates an example flow-chart of method operations of sensor signal correction using LMW;

FIG. 4 illustrates an example block diagram of an application server communicatively coupled with the wearable electronic device shown in FIG. 1A and FIG. 1B; and

FIG. 5 is a flow-chart of method operations for correcting a plurality of sensor streams in real time.

Corresponding reference characters indicate corresponding parts throughout the several views of the drawings. Although specific features of various examples may be shown in some drawings and not in others, this is for convenience only. Any feature of any drawing may be referenced or claimed in combination with any feature of any other drawing.

Some structural or method features may be shown in specific arrangements and/or orderings in the drawings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments, and, in some embodiments, it may not be included or may be combined with other features.

### DETAILED DESCRIPTION

Reference will now be made in detail to representative embodiments/aspects illustrated in the accompanying drawings. It should be understood that the following description is not intended to limit the embodiments to one preferred embodiment. On the contrary, it is intended to cover alternatives, modifications, and equivalents as can be included within the spirit and scope of the described embodiments as defined by the appended claims.

Embodiments described herein provide an apparatus and a method for real-time analysis of sensor data from one or more sensors included in a wearable electronic device. As described herein, the real-time analysis of sensor data is challenging due to artifacts introduced in the sensor data because of various noise sources and/or environmental factors including motion, humidity, and/or temperature, etc. Additionally, or alternatively, missing sensor data due to the environmental factors also makes real-time and accurate analysis of the sensor data a challenging task.

Embodiments described herein enable real-time analysis of the sensor data by removing artifacts and/or bridging a gap in the missing sensor data with sub-second latency. Embodiments disclosed herein addresses the problems mentioned herein, and also ensure that sensor data from each of a plurality of sensors is properly synchronized for real time adaptation. Synchronized sensor data thus curated for removal of artifacts and/or missing sensor data may be provided to other applications for presenting to the user. The embodiments may be performed by at least one processor of the wearable device. Additionally, or alternatively, embodiments may be performed by at least one processor of a computing device communicatively coupled with the wearable electronic device based upon sensor data received from one or more sensors of the plurality of sensors positioned at or within the wearable electronic device.

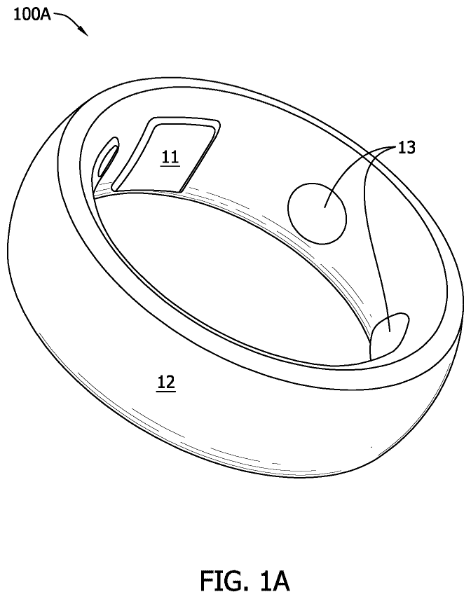

FIG. 1A illustrates an example wearable electronic device 100A. The wearable electronic device 100 may be worn by a user or secured to the user. By way of an example, the wearable electronic device may include, but is not limited to, a wearable computer, a wearable watch, a wearable communication device, a wearable media player, a wearable ring, a wearable health monitoring device, and the like. In one example, the wearable electronic device 100A may be a wearable electronic device including multiple functionalities such as time keeping, health monitoring, sports monitoring, medical monitoring, communications, navigation, computing operations, monitoring a user's physiological and/or biological signals and providing health-related information based on those signals, and/or communicating with other electronic devices or services in a wired or wireless manner. The wearable electronic device 100A may also provide alerts to the user, which may include one or more of audio, haptic, visual and/or other sensory output. The wearable electronic device 100A may also display data on a display device of the wearable electronic device 100A and acquire sensor data form one or more sensors positioned at or within the wearable electronic device 100A.

The wearable electronic device 100A includes a device body 11 including a housing that carries, encloses, and supports both externally and internally various components (including, for example, integrated circuit chips and other circuitry) to provide computing and functional operations for the wearable electronic device 100A. The components may be disposed on the outside of the housing, partially within the housing, through the housing, completely inside the housing, and the like. The housing may, for example, include a cavity for retaining components internally, holes or windows for providing access to internal components, and various features for attaching other components. The housing may also be configured to form a water-resistant or water-proof enclosure for the device body 11. For example, the housing may be formed from as a single unitary body and the openings in the unitary body may be configured to cooperate with other components to form a water-resistant or water-proof barrier. By way of a non-limiting example, the device body 11 may include components such as, but not limited to, processing units, memory, display, sensors, biosensors, speakers, microphones, haptic actuators, batteries, and so on. The wearable electronic device 100A may also include a band 12 or strap or other means for attaching to a user. In the example wearable electronic device 100A, the band 12 has an annular shape with an aperture to receive a finger of a subject user.

Additionally, the device body 11 may have one or more contact areas 13 for cognitive stress measurement for the user. By way of an example, the one or more contact areas may be provided as one or more buttons on the sides of the device body 11. Additionally, or alternatively, one or more EDA sensors, one or more temperature sensors, and/or one or more PPG sensors may be positioned on the bottom of the device body 11 such that these sensors come into contact with skin of the subject user or face the skin on the body for measuring various physiological and/or biological events.

FIG. 1B illustrates components of the wearable electronic device 100B according to an example of this disclosure. As shown in FIG. 1B, the wearable electronic device 100B includes an emitter 10 and detector 20, which are coupled to a processor 30. The processor 30 is coupled to a non-transitory storage medium 40. The wearable electronic device 100B is coupled to an output device 90. The wearable electronic device 100B is the wearable electronic device 100A shown in FIG. 1A.

The emitter 10 delivers light to a tissue and the detector 20 collects the optically attenuated signal that is backscattered from the tissue. In at least one example, the emitter 10 can be configured to emit at least three separate wavelengths of light. In another example, the emitter 10 may be configured to emit at least three separate bands or ranges of wavelengths. In at least one example, the emitter 10 may include one or more light emitting diodes (LEDs). The emitter 10 may also include a light filter. The emitter 10 may include a low-powered laser, LED, or a quasi-monochromatic light source, or any combination thereof. The emitter may emit light ranging from infrared to ultraviolet light. As indicated above, the present disclosure uses Near-Infrared Spectroscopy (NIRS) as a primary example and the other types of light can be implemented in other examples and the description as it relates to NIRS does not limit the present disclosure in any way to prevent the use of the other wavelengths of light.

The data generated by the detector 20 may be processed by the processor 30, such as a computer processor, according to instructions stored in the non-transitory storage medium 40 coupled to the processor. The processed data can be communicated to the output device 90 for storage or display to a user. The displayed processed data may be manipulated by the user using control buttons or touch screen controls on the output device 90 or on the device body 11.

The wearable electronic device 100B may include an alert module 50 configured to generate an alert. The processor 30 may send the alert to the output device 90 or the alert module 50 may send the alert directly to the output device 90. In at least one example, the wearable electronic device 100B may be configured so that the processor 30 is configured to send an alert to the output device 90 without the device including an alert module 50.

The alert may provide notice to a user, via a speaker or display on the output device 90, of a change in biological indicator conditions or other parameter being monitored by the wearable electronic device 100B, or the alert may be used to provide an updated biological indicator level to a user. In at least one example, the alert may be manifested as an auditory signal, a visual signal, a vibratory signal, or combinations thereof. In at least one example, an alert may be sent by the processor 30 when a predetermined biological indicator event occurs during a physical activity.

In at least one example, the wearable electronic device 100B may include a Global Positioning System (GPS) module 60 configured to determine geographic position and tagging the biological indicator data with location-specific information. The wearable electronic device 100B may also include a thermistor 70 and an IMU 80. The IMU 80 may be used to measure, for example, gait performance of a runner or pedal kinematics of a cyclist, as well as physiological parameters of a user during a physical activity. The thermistor 70 and IMU 80 may also serve as independent sensors configured to independently measure parameters of physiological threshold. The thermistor 70 and IMU 80 may also be used in further algorithms to process or filter the sensor signals data.

In some embodiments, artifacts or noise in sensor data may be removed based upon a combination of multiple sensor inputs for updating or modifying sensor signal acquisition parameters at the hardware level for obtaining corrected (or noise and/or artifacts free) sensor signals in real time (or with sub-second latency). Additionally, a gap in the sensor signal data is filled using an algorithm for real time interpolation and/or correction for unnatural changes in the sensor signal data. The gap in the sensor signal data may generally occur due to high acceleration, hardware anomalies or flag conditions, and/or changes in drive voltage, etc. By way of an example, the changes in drive voltage causing a gap in the sensor signal data may be abrupt and/or beyond threshold values.

### Real Time Gap Interpolation

In an example embodiment, real time gap interpolation is performed using two high-level steps: (i) identifying in real time when a gap has occurred; and (ii) adaptive interpolation across the gap. These steps are performed using a histogram that is maintained in real time such that a determination of expected values of sensor signal data from a sensor can be made.

In a case where a drift in sensor signal values is expected, checking for a change in a baseline sensor signal may not identify whether a gap or anomaly in the sensor signal occurred. Furthermore, sudden changes in the sensor signal occurring on a temporary basis generally do not constitute true gaps or deviations that would require or trigger interpolation. Additionally, once a gap or deviation is detected, it is generally not known for how long the gap or deviation will last for. Accordingly, extrapolating the sensor signal in real time is not a trivial task. Additionally, based upon a total number of raw sensor signals that are received before the gap or deviation is detected, an optimal extrapolation method that may be applied over the gap may vary.

Accordingly, in some embodiments, the sensor signal values of previously received sensor signals may be modified or updated to match more closely with the most recent sensor signal values. In other words, an history of previously received and/or reported sensor signal values may be revised to obtain a more accurate current result. In real time systems, the signal must be compensated on-the-fly as compared to other systems that are able to operate in an off-line or after recording context.

### Detecting Gaps and Deviations

Gaps in the sensor signal data may be because of missing sensor data, while deviations in the sensor signal data may be because of missing or bad data, where bad data corresponds with noise and/or environment artifacts. Detecting missing data region is associated with recognizing a deviation in a rate of sensor signals from a normal or desired rate of sensor signals. In as much as the rate of sensor signals is typically uniform, a histogram and/or a distribution of sensor signal rates may not be required. However, deviations in the expected sensor signal values returned by the sensor may require a distribution of sensor signal values and their derivatives to be maintained in real time. The distribution of sensor signal values may be used to determine (i) when interpolation should occur; 2) when overt sensor value manipulation/correction needs to be applied.

In an example embodiment, the distribution of sensor signal values may follow a live memory weighting (LMW), as described herein. By way of a non-limiting example, upon reading each new sensor signal value, for example, of a voltage, current, resistance, and/or capacitance, a histogram may be generated for the value and its derivative according to the following. If the new sensor signal value is determined to lie within the bounds of the histogram, a count is incremented for the appropriate bin of the histogram corresponding to the new sensor signal value. However. If the new sensor signal value is determined to lie outside of the bounds of the histogram, the histogram is recreated to include the out-of-bounds values by adjusting the bin edges and recounting existing values.

If the new sensor signal value is determined to lie outside of the minimum and maximum values of the current distribution, the histogram may be updated to correct for skewness. In other words, if the new sensor signal value is determined to be far greater than the largest value in the current distribution, it may happen that almost all the existing values are landing in a single bin, with many empty bins before a final bin with that single sensor signal value that far greater than the largest value in the current distribution. Accordingly, skewness of the histogram is corrected by knocking some of the values into neighboring bins using slight adjustments to bin edges.

Additionally, a cutoff value for the current histogram distribution may be determined for an outlier. Since the histogram's bound, which are related to the width of the distribution, are constantly changing, it is required that distribution “tails” are not decaying exponentially. Accordingly, in some embodiments, a deviant score for each value in the histogram may be computed by comparing the histogram value to the overall size of the histogram such that the adjusted histogram is basically a weighted distribution except that bins below a specified size tolerance are skipped altogether. Since histograms may not be normally distributed, it is possible for large peaks in the distribution may occur at multiple locations. After sorting the bins by probability, the cutoff value is determined as the first bin whose weighted probability falls below the threshold value. The adjusted threshold is selected empirically for each sensor signal of interest.

In some embodiments, before a new sensor signal value is processed, sufficient statistical information is gathered for the distribution before making decisions about how the new sensor signal to be processed. Sufficient statistical information is received for the period referenced herein as a “deviant burn-in period.” By the time, the “deviant burn-in period” has elapsed, sufficient number of samples (or sensor signals) are present in the distribution, and the new samples (or sensor signals) may be identified as being deviant or not.

Accordingly, the disclosed embodiments describe a real time deviant detection in which a sample may be classified as a deviant at the time of its acquisition and the same sample may be classified as a regular sample due to the statistics of the distribution being changed or updated. This illustrates why real time deviant detection is much more difficult than after-the-fact (offline) processing for deviant detection.

### Interpolating Across Gaps and Deviations

If a deviation or gap is detected in the acquired sensor signals, a real time interpolation needs to be performed. In connection with the real time buffering disclosed above during the “deviant burn-in period,” samples (or sensor signals) from two or more sensors are combined and reported as “windows” in time. The windows from two or more sensors have the same slice in time, or the windows from each of the two or more sensors have a respective explicit start and stop times. Additionally, each window has at least a certain minimum number of samples.

As the window strides through time, and because the stride value may be different for the window size, the same sections in time may be returned multiple times as part of a different slice in time. Since the same slice in time is returned multiple times, history for those samples may be revised before returning to the next window. Thus, as new samples become available, interpolation or signal corrections may be made until the interpolation or correction is in the past relative to the current window being returned.

When a gap in samples is first encountered or detected, a spline is created based upon a trajectory of the existing points or the current samples. The size of the gap controls knot weights or a smoothing parameter such that there is a higher probability of introducing curvature into the sensor signal for a smaller gap. For a larger gap, the smoothing parameter or know weights may be aggressively selected such that extrapolation errors across large gaps are avoided or minimized. Once samples or (sensor signals) are again available, based upon on the gap size, the interpolation steadily improves until the gap is cleanly interpolated. During the gap traversal, the end of the interpolation is “guessing” into the gap and may float across multiple values as the gap increases and the smoothing parameter decreases.

In an example embodiment, the spline is created based upon mirrored boundary conditions duplicating the last known samples before the gap, in reverse order, while maintaining the gap in time between those samples. The number of samples reverse appended is a padding parameter that is chosen relative to the size of the circular buffer being used. For example, a circular buffer of 200 samples may use a reverse append pad of 20 samples. However, once new samples on the other side of the gap are available, reverse append pads are not used because the past samples may not smoothly connect to the new ones.

In some embodiments, an order of the splines may be reduced if the number of samples available is too low to correctly fit a higher-order spline. Once the interpolation has been calculated, the baseline may be removed so that continuity in the first and second derivatives is obtained for a clean continuation relative to the previously reported windows. By way of a non-limiting example, the baseline removal may be accomplished by subtracting a least squares fit of the interpolation from a least squares fit to the existing data before the interpolation. With such a baseline correction, artifacts are introduced into the signal that may adversely affect algorithms consuming the raw sensor data. These interventions happen before reporting the sensor data to algorithms; thus, some semblance of reality is maintained. For example, as this is real life data, the curvature of a signal is expected to be continuous.

In some embodiments, when sensor signals are received from multiple different sensors, the region to interpolate over may come from deviant analysis of different signals from the multiple different sensors. By way of a non-limiting example, an electrodermal activity (EDA) signal may use real time interpolation over regions of high acceleration to reduce the noise in the EDA signal. Similarly, anomalies in the hardware exposed as flagged regions may induce interpolation across multiple signals.

### Live Memory Weighting (LMW)

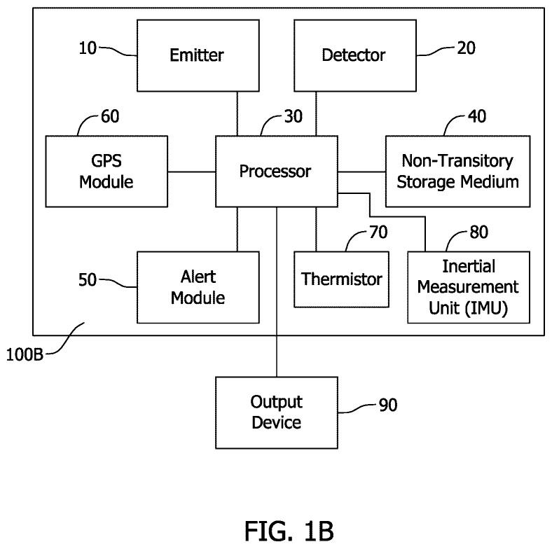

Live memory weighting (LMW), as described herein, is a real time signal correction technique that leverages the deviant histograms described herein to provide signal modification when unnatural and/or anomalous behavior is detected in the sensor signal. The LMW performs a correction while maintaining a natural sensor signal that is well-behaved across a wide range of data types and values. An example LMW is illustrated in FIG. 2.

FIG. 2 illustrates an example of LMW 200 in real time. Due to deviant scores detected at the large peak, LMW 200 may exponentially damp that peak and seamlessly connect with the regular samples on the other side. The sensor signal correction is described in detail herein. When the sensor signal does not significantly deviate from its expected behavior, the LMW correction is not applied. The LMW allows unnatural deviations from signal behavior to be corrected in real time by updating the deviant histograms and obtaining a deviant score, as described herein.

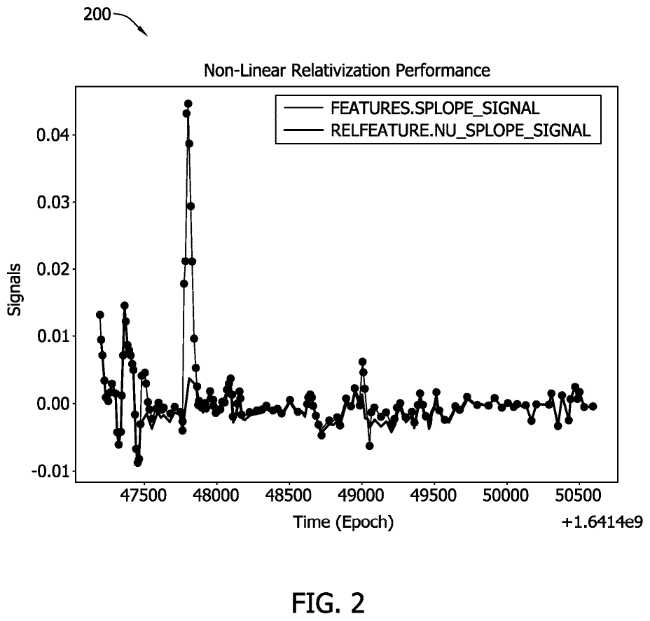

FIG. 3 illustrates an example flow-chart 300 of method operations of sensor signal correction using LMW, according to embodiments described herein. The method operations include computing 302 a naturalness score. The naturalness score is directly related to the deviant score but provides an asymmetrical handling of rises versus falls. This is because physiological and/or biological signals of a sensor signal generally has an asymmetry between natural rises and falls. For example, a rise of the sensor signal associated with the physiological and/or biological signals may resemble a hyperbolic sine function while a fall of the sensor signal may more closely resemble an exponential decay.

The naturalness score is computed by comparing the deviant score for the value and its derivative to the cutoff value computed from their respective histograms. If the deviant lies beyond the cutoff (meaning that it is considered deviant in the sense of the previous section), then we construct a subset of the signal at the current value using an array of ‘[previous, previous, current, current]’, where “previous” is the value from the last round of LMW, and current is the current value. This creates a step-function signal between the previous value and the current value.

Due to the “step function” shape of the target signal, a variety of transfer functions can be used, depending on the signal of interest. For example, a sigmoid function, hyperbolic tangent, linear, Gaussian, or any other function with the characteristic “S” shape.

The method operations include exponentially damping 304 the sensor signal based upon the computed 302 naturalness score when the signal is unnatural. The unnatural signal may be off by orders of magnitude and using exponential damping 304 even unsightly deviations may be transformed or converted to reasonable ones. The exponential damping 304 is performed according to a damping parameter. A value of the damping parameter is determined or selected based upon the computed 302 naturalness score.

Exponential damping 304 is applied using a, the current signal value, the previous signal value, and scaling parameters. Scaling parameters for rises and falls may be separate parameters.

By way of a non-limiting example, in an example decay function, using normalized dy derivatives along with the slope parameter of the fit may allow a highly customized damping behavior in comparison to hyperbolic, power law, and/or non-linear combinations of composed functions that each or together produce a damping effect on the parameters.

The method operations include applying 306 a drift correction to the sensor signal. Due to the asymmetry in rises and falls of the sensor signals, unnaturalness from a rise may require heavy exponential damping 304 while the fall may require a different level of damping 304, or no damping at all. For example, in EDA signals, washing hands introduces unnatural spikes in EDA due to excessive skin moisture. However, the exponential decay due to evaporation of that moisture after washing hands is completely natural. From a wearable electronic device or biometrics perspective, the unnatural rise and fall are removed without causing drift in the sensor signal that was not originally there. Further, the baseline of the EDA signal returns to normal after the evaporation event is completed.

The drift correction is applied 306 based upon a computed momentum of the sensor signal. The momentum is computed based upon the last several values (e.g., 30 values or other predetermined number of values) of the drift correction.

In addition to the momentum, the drift correction is applied 306 based upon jitter computed using the last several values of the original sensor signal. In at least one example, the asymmetry between rises and falls includes repeated rise corrections followed by uncorrected falls that can result in signal drift well below the correct baseline of the signal. However, to fix this issue, LMW technique, as described herein, is applied 308. The applied 308 LMW technique provides sophisticated drift correction.

Accordingly, technical benefits of the embodiments described herein addresses requirements of sub-second latency, while removing artifacts introduced into signals from motion or other environmental factors that cannot be removed using regular signal processing techniques. The embodiments described herein modify the sensor samples in real time to remove the anomalous or unnatural behaviors caused by the environment, and to return the sensor signal values to values that are immediately consumable by applications. Since the firmware has access to hardware flags and other data at the point of acquisition, which regions need to be interpolated across in real time to provide corrections are accurately identified.

Real time corrections disclosed herein allow on-the-fly removal based on signal deviation distributions coupled with sophisticated drift control after adaptive, exponential damping of signal anomalies has been applied. Together with the real time gap interpolation disclosed herein, these methods allow acquisition of clean sensor data from multiple sensor sources simultaneously with immense practical use across the wearable electronic device domain. Because the methods are generalized to the distribution of sensor signal values, the embodiments may be applied in multiple circumstances.

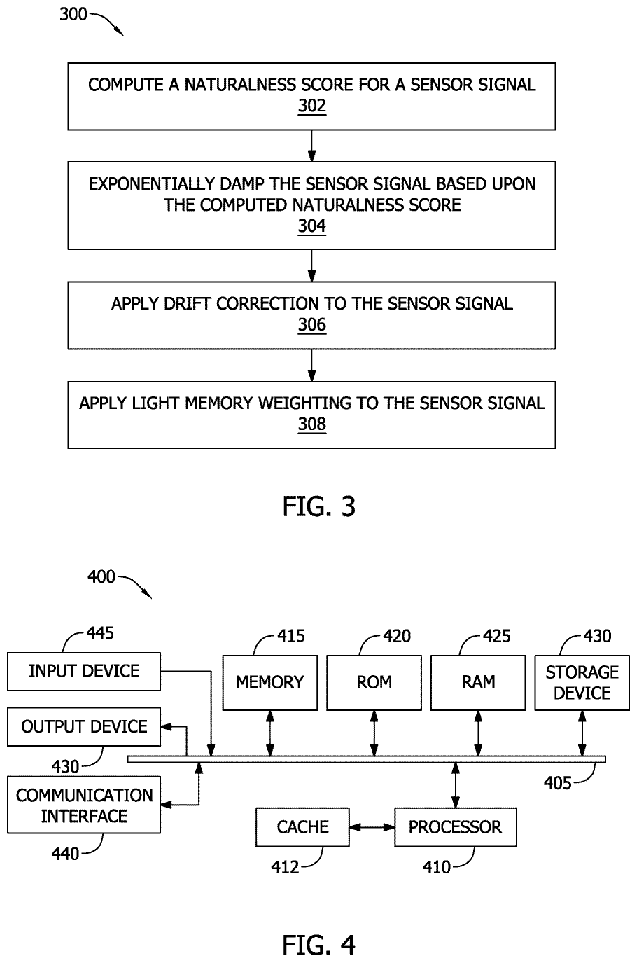

FIG. 4 illustrates an example block diagram of an application server 400 communicatively coupled with the wearable electronic device shown in FIG. 1A and FIG. 1B. The application server 400 can implement various techniques, processes, functions, or methods described herein. The components of the application server 400 are shown in electrical communication with each other using a connection 405, such as a bus. The example application server 400 includes a processing unit (CPU or processor) 410 and an application server connection 405 that couples various application server components, including application server memory 415, such as a read only memory (ROM) 420 and a random-access memory (RAM) 425, to processor 410.

Application server 400 can include a cache 412 of high-speed memory connected directly with, in close proximity to, or integrated as part of processor 410. Application server 400 can copy data from memory 415 and/or storage device 430 to cache 412 for quick access by processor 410. In this way, cache 412 can provide a performance boost that avoids processor 410 delays while waiting for data. These and other modules can control or be configured to control processor 410 to perform various actions. Other application server memory 415 may be available for use as well. Memory 415 can include multiple different types of memory with different performance characteristics. Processor 410 can include any general-purpose processor, central processing unit (CPU), or graphics processing unit (GPU) in combination with a hardware or software provision configured to control processor 410 and stored in storage device 430, as well as any special-purpose processor where software instructions are incorporated into the processor design. Processor 410 may be a self-contained system, containing multiple cores or processors, a bus, memory controller, cache, etc. A multi-core processor may be symmetric or asymmetric.

Storage device 430 is a non-volatile memory and can be one or more of a hard disk or other types of computer readable media that can store data that are accessible by a computer, such as a magnetic cassette, flash memory card, solid state memory device, digital versatile disk, cartridge, RAM 425, ROM 420, or hybrids thereof. Memory 415 or storage device 430 can include software, code, firmware, etc., for controlling processor 410. Other hardware or software modules are contemplated. Memory 415 and storage device 430 are connected to application server connection 405. In one aspect, a hardware module that performs a particular function can include the software component stored in a computer-readable medium in connection with the necessary hardware components, such as processor 410, application server connection 405, and so forth, to carry out the function. In the example embodiment, processor 410 may be programmed by encoding an operation or function using one or more executable instructions and providing the executable instructions in memory 415 or storage device 430.

In operation, application server 400 may be a client computing device. The application server 400 may be a computer that executes computer-executable instructions embodied in one or more computer-executable components stored on one or more computer-readable media to implement aspects of the disclosure described or illustrated herein. The order of execution or performance of the operations in embodiments of the disclosure illustrated and described herein is not essential, unless otherwise specified. That is, the operations may be performed in any order, unless otherwise specified, and embodiments of the disclosure may include additional or fewer operations than those disclosed herein. For example, it is contemplated that executing or performing a particular operation before, contemporaneously with, or after another operation is within the scope of aspects of the disclosure.

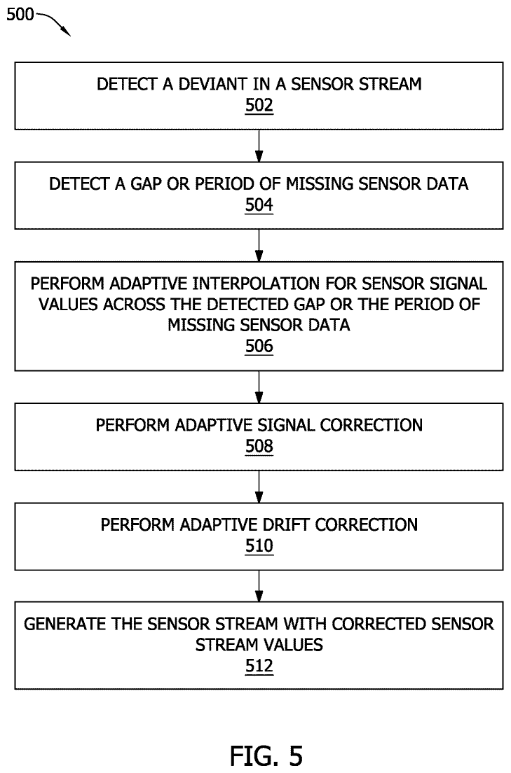

FIG. 5 is a flow-chart 500 of method operations for correcting a plurality of sensor streams in real time. The method operations may be performed by the wearable electronic device 100A or 100B, and/or a client computing device or an application server showed in FIG. 4. The method operations include detecting 502 a deviant in a sensor stream of the plurality of sensor streams based upon a distribution of sensor signal values in the sensor stream. As described herein, before a new sensor signal value is processed, sufficient statistical information is gathered for the distribution before making decisions about how the new sensor signal to be processed. Sufficient statistical information is received for the period referenced herein as a “deviant burn-in period.” By the time the “deviant burn-in period” has elapsed, sufficient number of samples (or sensor signals) are collected or gathered, and the new one or more samples (or one or more sensor signals) may be identified as being deviant or not, based upon the sufficient number of samples collected or gathered, at the time of their acquisition. By way of a non-limiting example, the deviant is detected using weighted histograms with sensor-specific cutoff values.

The method operations include detecting 504 a gap or a period of missing sensor data based upon deviation in a sensor signal sample rate. As described herein, gaps in the sensor signal data may be because of missing sensor data. Detecting missing data region or the period of missing sensor data is based upon a recognized deviation in a rate of sensor signals from a normal or desired rate of sensor signals. While the rate of sensor signals is typically uniform, a histogram and/or a distribution of sensor signal rates may not be required. However, a deviation in the expected sensor signal values returned by the sensor may be recognized based upon a distribution of sensor signal values and their derivatives to be maintained in real time.

The method operations include performing 506 an adaptive interpolation for sensor signal values across the detected gap or the period of missing sensor data. As described herein, real time gap interpolation is performed for the detected gap using a histogram that is maintained in real time such that a determination of expected values of sensor signal data from a sensor can be made. Interpolation across the detected gaps is described in detail herein, and accordingly those details are not repeated here for the sake of brevity. As described herein, the adaptive interpolation, when performed, changes boundary conditions dependent on a gap size and/or causes an increase in smoothness with gap size until a valid value is obtained after the gap. Additionally, or alternatively, the adaptive interpolation causes the gap interpolation to change with window stride creating a revised history of the sensor signal as more sensor signal data becomes available. Additionally, or alternatively, the adaptive interpolation is performed using interpolating splines, polynomials, orthogonal basis functions, wavelet recomposition, Fourier extrapolation, or statistical random draws from empirical or common distributions.

Additionally, the method operations include performing 508 adaptive signal correction by suppressing derivative values of the detected deviant. In the present disclosure, how to handle a new sensor signal value based upon bounds of a histogram, a minimum value and a maximum value of a current distribution of the histogram, skewness of the histogram, and/or a cutoff value for the current distribution of the histogram is described detail, and accordingly those details are not repeated for the sake of brevity. By way of a non-limiting example, the signal correction may be based upon a decaying exponential function whose strength depends on the signal derivative or on a sigmoidal fit to the signal's values. Additionally, or alternatively, the signal correction may be based upon a decaying exponential function whose strength varies as a non-linear combination of the signal value and its derivatives, and parameters derived from fits to standard transfer functions such as rectified linear unit, sigmoid, step function, Gaussian, or hyperbolic tangent.

The method operations include performing 510 adaptive drift correction to handle asymmetries in a sensor signal during a rise and fall of the sensor signal. Adaptive drift correction using momentum, jitter, and LMW technique is described herein in detail, and therefore those details are not repeated for the sake of brevity. By way of a non-limiting example, the adaptive drift correction may be based upon drift momentum and/or signal jitter computed for a predetermined number of previous sensor signal samples. The sensor stream is thus generated 512 with corrected sensor signal value. The corrected sensor stream is made available to other applications for processing of the sensor signal data. Additionally, the method operations include performing a distribution skewness correction for a warped histogram. The sensor signal values of the sensor streams are stored in a histogram.

As used herein, the phrase “at least one of” preceding a series of items, with the term “and” or “or” to separate any of the items, modifies the list as a whole, rather than each member of the list. The phrase “at least one of” does not require selection of at least one of each item listed; rather, the phrase allows a meaning that includes at a minimum one of any of the items, and/or at a minimum one of any combination of the items, and/or at a minimum one of each of the items. By way of example, the phrases “at least one of A, B, and C” or “at least one of A, B, or C” each refer to only A, only B, or only C; any combination of A, B, and C; and/or one or more of each of A, B, and C. Similarly, it may be appreciated that an order of elements presented for a conjunctive or disjunctive list provided herein should not be construed as limiting the disclosure to only that order provided.

One may appreciate that although many embodiments are disclosed herein, that the operations and steps presented with respect to methods and techniques described herein are meant as exemplary and accordingly are not exhaustive. One may further appreciate that alternate step order or fewer or additional operations may be required or desired for particular embodiments.

Although the disclosure herein is described in terms of various exemplary embodiments and implementations, it should be understood that the various features, aspects, and functionality described in one or more of the individual embodiments are not limited in their applicability to the particular embodiment with which they are described, but instead can be applied, alone or in various combinations, to one or more of some embodiments, whether or not such embodiments are described and whether or not such features are presented as being a part of a described embodiment. Thus, the breadth and scope of the present description should not be limited by any of the exemplary embodiments described herein but is instead defined by the claims herein presented.

What is claimed is:

1. A computer-implemented method for correcting a plurality of sensor streams in real time, the computer-implemented method comprising:
detecting a deviant in a sensor stream of the plurality of sensor streams based upon a distribution of sensor signal values in the sensor stream;
detecting a gap or a period of missing sensor data based upon deviation in a sensor signal sample rate;
performing adaptive interpolation for sensor signal values across the detected gap or the period of missing sensor data;
performing adaptive signal correction by suppressing derivative values of the detected deviant;
performing adaptive drift correction to handle asymmetries in a sensor signal during a rise and fall of the sensor signal; and
generating the sensor stream with corrected sensor signal values.

2. The computer-implemented method of claim 1, further comprising performing a distribution skewness correction for a warped histogram, wherein the sensor signal values of the sensor stream are stored in a histogram.

3. The computer-implemented method of claim 1, wherein detecting the deviant in the sensor stream comprises detecting the deviant using weighted histograms with sensor-specific cutoff values.

4. The computer-implemented method of claim 1, wherein performing the adaptive drift correction comprises performing drift correction based upon drift momentum computed for a predetermined number of previous sensor signal samples to adjust the drift correction.

5. The computer-implemented method of claim 4, further comprising performing drift correction based upon signal jitter computed for a predetermined number of previous sensor signal samples to adjust the drift correction.

6. The computer-implemented method of claim 1, wherein the signal correction is a decaying exponential function whose strength depends on a signal derivative.

7. The computer-implemented method of claim 1, wherein the signal correction is based upon a decaying exponential function whose strength depends on a sigmoidal fit to the signal's values.

8. The computer-implemented method of claim 1, wherein the signal correction is based upon a decaying exponential function whose strength varies as a non-linear combination of the signal value and its derivatives, and parameters derived from fits to standard transfer functions such as rectified linear unit, sigmoid, step function, Gaussian, or hyperbolic tangent.

9. The computer-implemented method of claim 1, wherein performing the adaptive interpolation comprises performing gap interpolation using interpolating splines, polynomials, orthogonal basis functions, wavelet recomposition, Fourier extrapolation, or statistical random draws from empirical or common distributions.

10. The computer-implemented method of claim 9, wherein performing the adaptive interpolation causes the gap interpolation to change with window stride creating a revised history of the sensor signal as more sensor signal data becomes available.

11. The computer-implemented method of claim 1, wherein performing the adaptive interpolation changes boundary conditions dependent on a gap size.

12. The computer-implemented method of claim 1, wherein performing the adaptive interpolation causes an increase in smoothness with gap size until a valid value is obtained after the gap.

13. A system for correcting a plurality of sensor streams in real time, the system comprising:
at least one non-transitory memory storing instructions; and
at least one processor communicatively coupled with the at least one non-transitory memory and configured to execute the instructions to perform operations comprising:
detecting a deviant in a sensor stream of the plurality of sensor streams based upon a distribution of sensor signal values in the sensor stream;
detecting a gap or a period of missing sensor data based upon deviation in a sensor signal sample rate;
performing adaptive interpolation for sensor signal values across the detected gap or the period of missing sensor data;
performing adaptive signal correction by suppressing derivative values of the detected deviant;
performing adaptive drift correction to handle asymmetries in a sensor signal during a rise and fall of the sensor signal; and
generating the sensor stream with corrected sensor signal values.

14. The system of claim 13, wherein the operations further comprising performing a distribution skewness correction for a warped histogram, wherein the sensor signal values of the sensor stream are stored in a histogram.

15. The system of claim 13, wherein detecting the deviant in the sensor stream comprises detecting the deviant using weighted histograms with sensor-specific cutoff values.

16. The system of claim 13, wherein performing the adaptive drift correction comprises performing drift correction based upon drift momentum and signal jitter computed for a predetermined number of previous sensor signal samples to adjust the drift correction.

17. The system of claim 13, wherein the signal correction is based upon a decaying exponential function whose strength depends on a signal derivative, on a sigmoidal fit to the signal's values, and/or a non-linear combination of the signal value and its derivatives, and parameters derived from fits to standard transfer functions such as rectified linear unit, sigmoid, step function, Gaussian, or hyperbolic tangent.

18. The system of claim 13, wherein performing the adaptive interpolation comprises performing gap interpolation using interpolating splines, polynomials, orthogonal basis functions, wavelet recomposition, Fourier extrapolation, or statistical random draws from empirical or common distributions.

19. The system of claim 18, wherein:
performing the adaptive interpolation changes boundary conditions dependent on a gap size;
performing the adaptive interpolation causes an increase in smoothness with gap size until a valid value is obtained after the gap; and/or
performing the adaptive interpolation causes the gap interpolation to change with window stride creating a revised history of the sensor signal as more sensor signal data becomes available.

20. A non-transitory computer-readable media storing instruction thereon, which, when executed by at least one processor of at least one computing device, cause the at least one computing device to correct a plurality of sensor streams in real time by performing operations comprising:
detecting a deviant in a sensor stream of the plurality of sensor streams based upon a distribution of sensor signal values in the sensor stream;
detecting a gap or a period of missing sensor data based upon deviation in a sensor signal sample rate;
performing adaptive interpolation for sensor signal values across the detected gap or the period of missing sensor data;
performing adaptive signal correction by suppressing derivative values of the detected deviant;
performing adaptive drift correction to handle asymmetries in a sensor signal during a rise and fall of the sensor signal; and
generating the sensor stream with corrected sensor signal values.
